(19) United States
(12) Patent Application Publication
(10) Pub. No.: US 2013/0268034 A1
(43) Pub. Date: Oct. 10, 2013

(54) PHOTOTHERAPY APPARATUS

(71) Applicants: Yuki Kawase, Kanagawa (JP); Yoshiteru II, Kanagawa (JP); Daisuke Okamura, Kanagawa (JP)

(72) Inventors: Yuki Kawase, Kanagawa (JP); Yoshiteru II, Kanagawa (JP); Daisuke Okamura, Kanagawa (JP)

(73) Assignee: PANASONIC CORPORATION, Osaka (JP)

(21) Appl. No.: 13/995,272

(22) PCT Filed: Apr. 2, 2012
(86) PCT No.: PCT/JP2012/002301

§ 371 Date: Jun. 18, 2013

(30) Foreign Application Priority Data
Apr. 7, 2011 (JP) 2011-085143
Apr. 7, 2011 (JP) 2011-085144
Jun. 21, 2011 (JP) 2011-136911

(51) Int. Cl. A61N 5/06

(52) U.S. Cl. CPC A61N 5/0613
USPC 607/91; 607/88

(57) ABSTRACT

Provided is a phototherapy apparatus capable of applying appropriate treatment. Specifically, the purpose of the present invention is to provide a phototherapy apparatus capable of easily supplying adequate treatment light even when a region to be treated is a bent region like finger joints of a patient having severe rheumatoid arthritis, thus making it possible to apply appropriate treatment. This phototherapy apparatus is configured to include: a light guide including a first surface on which an affected part is placed and a second surface on a back surface side of the first surface; a light source that outputs therapeutic light to enter an inside of the light guide body; and a light guide member that has softness and surface tackiness and is disposed on a part of the first surface.

### TECHNICAL FIELD

The present invention relates to a phototherapy apparatus.

### BACKGROUND ART

Phototherapy apparatuses, using, for example, infrared light (about 700 to 2500 nm) as therapeutic light, are used to alleviate pain due to chronic non-infectious inflammation in muscles and joints or to treat rheumatoid arthritis. These conventional phototherapy apparatuses are so configured as to apply therapeutic light from a light source onto a part to be treated (treatment part) in a non-contact manner either directly or via a light guide path (see, e.g., PTL 1). However, the intensity of the light used for such therapy is strong, and hence the direct entry of intense light to the eye of a patient is not desirable.

To cope with this problem, there has been proposed a configuration in which, in the state where therapeutic light from a light source is introduced into a light guide body, such as acrylic and glass, a treatment part is brought into contact with the surface of the light guide body (see, e.g., PTL 2). The therapeutic light introduced into the light guide body is applied to the treatment part which is in contact with the light guide body. That is, in the phototherapy apparatus proposed by PTL 2, in the state where the treatment part is not in contact with the surface of the light guide body, the therapeutic light introduced into the light guide body from the light source is prevented from being emitted to the outside of the light guide body, while, in the state where the treatment part is in contact with the surface of the light guide body, the therapeutic light introduced into the light guide body is applied to the treatment part which is in contact with the surface of the light guide body.

Thereby, unnecessary emission of therapeutic light from the light guide body is prevented, and entry of therapeutic light into the eye of a patient is prevented.

### CITATION LIST

### Patent Literature

### PTL 1

Japanese Patent Application Laid-Open. No. SHO 63-21069

### PTL 2

Japanese Patent Application Laid-Open No. 2009-95549

### SUMMARY OF INVENTION

### Technical Problem

When phototherapy is performed by using the phototherapy apparatus described in PTL 2, and when a treatment part is a bent part, such as a joint, the therapeutic light cannot be supplied to the treatment part unless the bent part is brought into contact with the surface of the light guide body. However, the phototherapy apparatus has a problem that, since it is difficult to bring such a bent part into contact with the surface of the light guide body, it is difficult to suitably supply the therapeutic light to the treatment part, and hence suitable therapeutic treatment cannot be performed.

For example, in the case of a patient with rheumatoid arthritis, especially in the case of a patient with severe rheumatoid arthritis, swelling of the joint continues for a long time, and thereby the joint capsule and the tissue around the joint are distorted or deformed. When the symptoms progress further, so-called joint deformation is caused. Joint deformation is a symptom in which the joint is bent because the articular surface is shifted and pulled by muscles around the joint with the progress of destruction of articular cartilage and joint subchondral bone due to synovitis. The symptom of joint deformation notably appears in finger joints.

Therefore, in the case of a patient with rheumatoid arthritis, therapeutic treatment needs to be applied to finger joints as treatment parts by bringing the finger joints into contact with the surface of the light guide body. For example, when the patient places a palm of a hand on the surface of the light guide body so as to receive therapeutic treatment of the hand and fingers, the patient must be required to perform an unnatural action to bring the finger joints into contact with the surface of the light guide body. Especially for a patient with severe rheumatoid arthritis, who has developed symptoms of joint deformation, such unnatural action causes pain. Further, in some patients with such symptoms, the finger joints are so deformed that it is difficult to bring the finger joints into contact with the surface of the light guide body. Such patients are difficult to bring the finger joints into contact with the surface of the light guide body. As a result, sufficient light cannot be supplied to the affected part, and thereby suitable therapeutic treatment becomes difficult to be performed.

On the other hand, it is also conceivable that the shape of the light guide body is made to match the shape of the affected part of the patient with rheumatoid arthritis so that the light guide body is brought into close contact with the bent part, such as finger joints, to enable sufficient therapeutic light to be supplied to the affected part. However, the states of deformation of finger joints of patients who have developed symptoms of joint deformation are different for each of the patients. For this reason, it is not realistic to prepare the light guide body for each shape of the affected parts. As a result, it becomes difficult to perform suitable therapeutic treatment.

Therefore, an object of the present invention is to provide a phototherapy apparatus which, even when a treatment part is a bent part, such as a finger joint of a patient with severe rheumatoid arthritis, can simply supply sufficient therapeutic light to the treatment part, and as a result, can provide suitable therapeutic treatment.

### Solution to Problem

To this end, a phototherapy apparatus according to the present invention is configured, by including: a light guide body that includes a first surface on which an affected part is placed, and a second surface on a back surface side of the first surface; a light source that outputs therapeutic light to enable the therapeutic light to enter an inside of the light guide body; and a light guide member that has softness and surface tackiness and is disposed on a part of the first surface. The present invention is featured in that the intended purpose is achieved by the above-described configuration.

### Advantageous Effects of Invention

In the phototherapy apparatus according to the present invention, the light guide member has softness, and hence is deformed according to the shape of a treatment part when the treatment part is merely disposed so as to be brought into contact with the light guide member disposed on the surface of the light guide body. Thereby, a bent part, such as a finger joint, can be brought into contact with the light guide member without requiring a patient to perform an unnatural action. Further, light having entered the inside of the light guide body can be supplied to the treatment part via the light guide member. As a result, sufficient therapeutic light can be supplied to a bent part, such as a finger joint, and thereby suitable therapeutic treatment can be performed.

Further, in a conventional phototherapy apparatus, when a hand is placed on a light guide body to supply light to a finger joint as a treatment part of therapeutic treatment of rheumatism, parts of the hand other than the finger joint are in contact with the light guide body, and hence are also supplied with light. On the other hand, in the phototherapy apparatus according to the present invention, the light guide member is disposed on the surface of the light guide body, and hence light can be intensively supplied to the treatment part. As a result, the energy efficiency of the phototherapy apparatus can also be improved. Further, even when therapeutic treatment needs to be intensively applied to the finger joint portion, therapeutic light can be highly efficiently supplied to the finger joint portion, and hence an improvement in therapeutic efficacy can also be expected.

Further, in the phototherapy apparatus according to the present invention, when an inexpensive and general-purpose material, such as silicone, resin, is used as the material of the light guide member, the light guide member can be used as a disposable member, and hence therapeutic treatment in consideration of sanitation can be performed.

### BRIEF DESCRIPTION OF DRAWINGS

FIG. 1 is a perspective view illustrating a phototherapy apparatus according to Embodiment 1 of the present invention;

FIG. 2 is a perspective view illustrating a use state of the phototherapy apparatus according to Embodiment 1 of the present invention;

FIG. 3 is a front perspective view of the phototherapy apparatus according to Embodiment 1 of the present invention;

FIG. 4 is a lateral perspective view of the phototherapy apparatus according to Embodiment 1 of the present invention;

FIG. 5 is a cross-sectional view of a light guide body of the phototherapy apparatus according to Embodiment 1 of the present invention;

FIG. 6 is a bottom view of the light guide body of the phototherapy apparatus according to Embodiment 1 of the present invention;

FIG. 7 is a perspective view of an auxiliary light guide body of the phototherapy apparatus according to Embodiment 1 of the present invention;

FIG. 8 is a top view of the auxiliary light guide body of the phototherapy apparatus according to Embodiment 1 of the present invention;

FIG. 9 is a side view of the light guide body provided with the auxiliary light guide body of the phototherapy apparatus according to Embodiment 1 of the present invention;

FIG. 10 is a perspective view illustrating an example of phototherapeutic treatment using the phototherapy apparatus according to Embodiment 1 of the present invention;

FIG. 11 is a bottom view illustrating the example of phototherapeutic treatment using the phototherapy apparatus according to Embodiment 1 of the present invention;

FIG. 12 is a view illustrating a state where therapeutic light is applied to an affected part by a conventional phototherapy apparatus;

FIG. 13 is a view illustrating a state where therapeutic light is applied to an affected part by the phototherapy apparatus according to Embodiment 1 of the present invention;

FIG. 14 is a front perspective view of a phototherapy apparatus according to Embodiment 3 of the present invention;

FIG. 15 is a lateral perspective view of the phototherapy apparatus according to Embodiment 3 of the present invention;

FIG. 16 is a perspective view of an auxiliary light guide body of the phototherapy apparatus according to Embodiment 3 of the present invention;

FIG. 17 is a cross-sectional view of the auxiliary light guide body of the phototherapy apparatus according to Embodiment 3 of the present invention;

FIG. 18 is a perspective view illustrating a state where the auxiliary light guide body is disposed on the surface of the light guide body of the phototherapy apparatus according to Embodiment 3 of the present invention;

FIG. 19 is a lateral cross-sectional view illustrating a state where the auxiliary light guide body is disposed on the surface of the light guide body of the phototherapy apparatus according to Embodiment 3 of the present invention;

FIG. 20 is a view illustrating phototherapeutic treatment of a hand using the phototherapy apparatus according to Embodiment 3 of the present invention;

FIGS. 21A and 21B are a view illustrating a change in the shape of a projecting section when a treatment part is placed on the auxiliary light guide body according to Embodiment 3 of the present invention;

FIG. 22 is a bottom view illustrating a state where a hand is placed on the auxiliary light guide body according to Embodiment 3 of the present invention;

FIG. 23 is a view illustrating supply of light to an affected part by using the phototherapy apparatus according to Embodiment 3 of the present invention;

FIG. 24 is a top view of a light guide body disposed on a base of a phototherapy apparatus according to Embodiment 4 of the present invention;

FIG. 25 is a top view illustrating a state where an auxiliary light guide body is disposed on the light guide body of the phototherapy apparatus according to Embodiment 4 of the present invention; and

FIG. 26 is a top view of the auxiliary light guide body according to Embodiment 4 of the present invention.

### DESCRIPTION OF EMBODIMENTS

Embodiments of a phototherapy apparatus according to the present invention will be described in detail with reference to accompanying drawings. Note that the phototherapy apparatus described below in each of Embodiments 1 to 4 is assumed to have, as an example, a configuration used for therapeutic treatment of finger joints of a patient with rheumatoid arthritis. Of course, the phototherapy apparatus according to the present invention is not limited to these.

### Embodiment 1

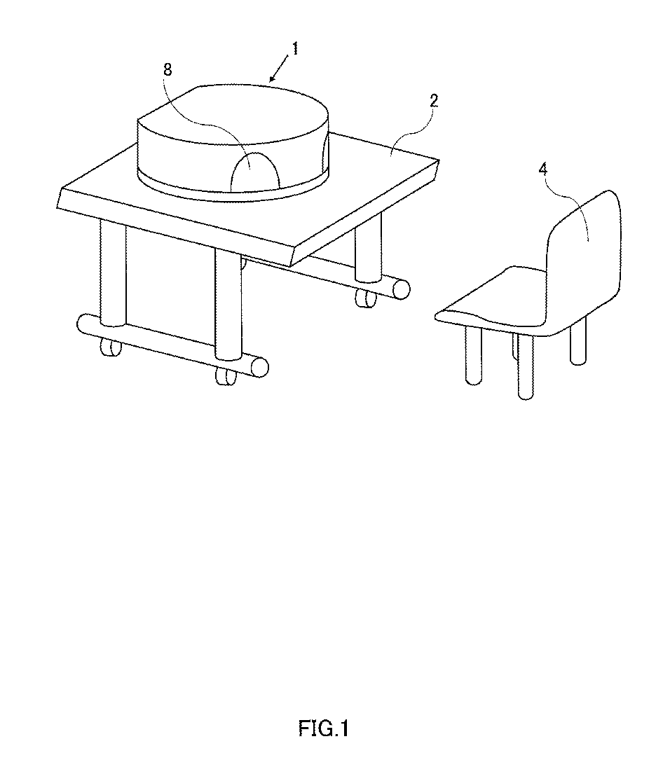

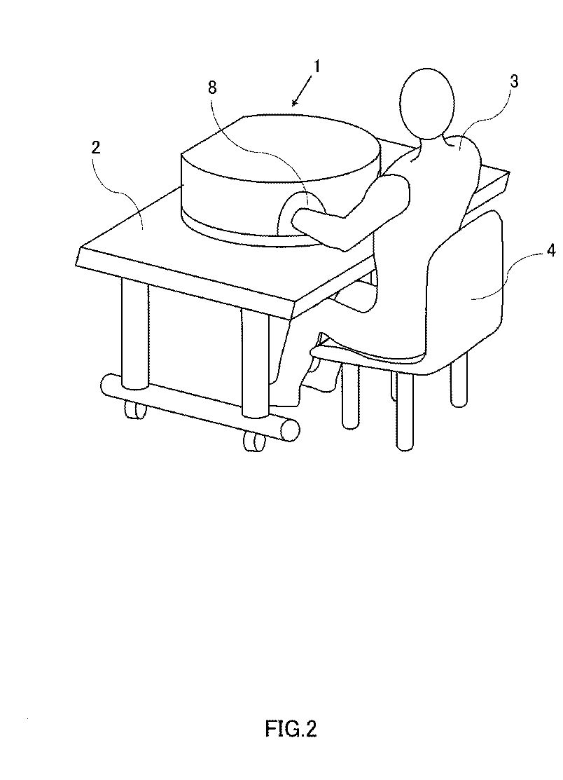

FIG. 1 is a perspective view illustrating a phototherapy apparatus according to Embodiment 1 of the present invention. Phototherapy apparatus 1 is mounted on desk 2. As shown in FIG. 2, patient 3 can sit on chair 4 and receive phototherapy using phototherapy apparatus 1 in his/her house or in a hospital.

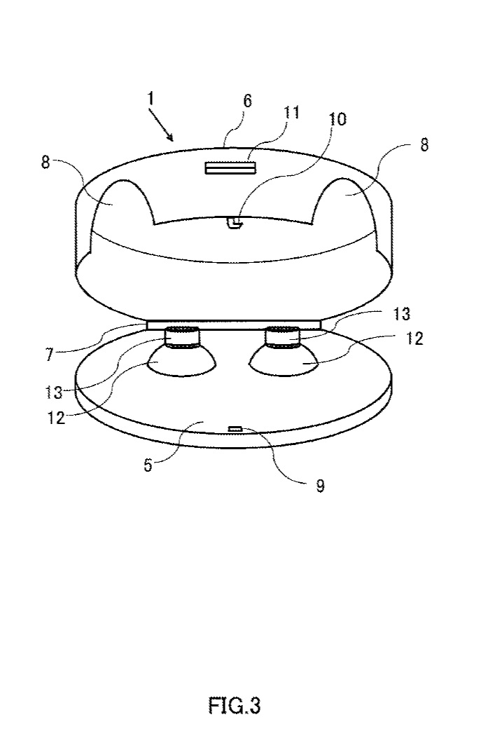

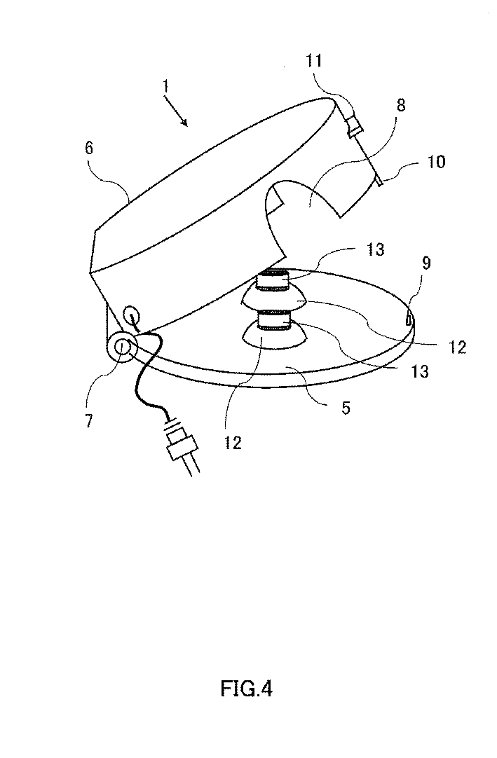

As shown in FIGS. 3 and 4, phototherapy apparatus 1 includes disc-like base 5 and lid 6. Lid 6 has a capped cylindrical shape whose lower surface is open. Lid 6 is connected to base 5 by hinge 7, and can cover the surface of base 5 in an openable and closable manner. When the surface of base 5 is covered by lid 6, intense light for use in phototherapeutic treatment can be prevented from directly entering the eye of the patient 3.

Left and right two affected part insertion ports 8 are provided on the front surface side of the outer peripheral surface of lid 6, that is, on the side opposite to hinge 7. Further, hook 10 is provided on the front side of the outer peripheral surface of lid 6 and between two affected part insertion ports 8. When hook 10 engages with engagement hole 9 of base 5, lid 6 is locked. Hook button 11 for releasing the lock is provided above hook 10.

On the surface of base 5, left and right two light guide bodies 12 are provided at a predetermined spacing. One or more detachable auxiliary light guide bodies 13 are disposed on a part of the outer surface (placement surface 14) of light guide body 12. The number and size of auxiliary light guide bodies 13 are freely determined according to the contents of therapeutic treatment, but in FIGS. 3 and 4, one auxiliary light guide body 13 is disposed on one light guide body 12.

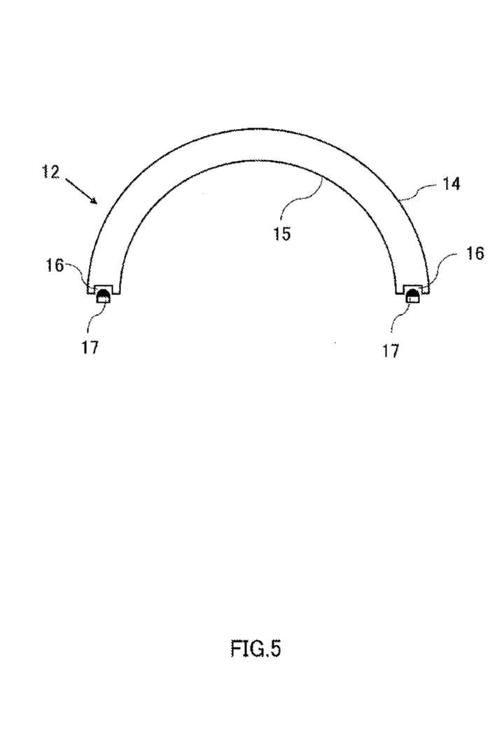

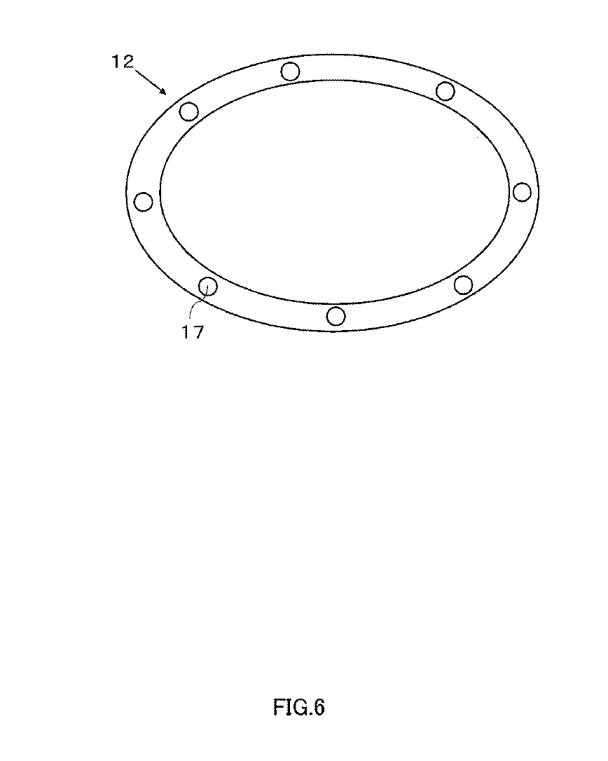

FIG. 5 is a cross-sectional view of light guide body 12. FIG. 6 is a bottom view of light guide body 12. As can be seen from FIGS. 5 and 6, light guide body 12 is formed in a hollow semi-spherical shape. Therefore, each of the outer and inner surfaces of light guide body 12 is also formed in a substantially semi-spherical shape. The outer surface of light guide body 12 is placement surface 14 on which an affected part (e.g., a hand and fingers) is placed, and the inner surface on the hack side of placement surface 14 is back-facing surface 15.

The space between placement surface 14 and back-facing surface 15 of light guide body 12 serves as a light guide path. The thickness of the light guide path (distance between placement surface 14 and back-facing surface 15) is substantially uniform. Placement surface 14 of light guide body 12 has a size sufficient for placing a hand and fingers thereon. Placement surface 14 of light guide body 12 is formed in a substantially semi-spherical shape, but as can be seen from FIG. 6, placement surface 14 needs not be formed in an exactly semi-spherical shape. Light guide body 12 illustrated in FIG. 6 is formed such that one diameter (lateral diameter in FIG. 6) is slightly larger than the other diameter (vertical diameter in FIG. 6) perpendicular to the one diameter. In this way, placement surface 14 of light guide body 12 is formed in a substantially semi-spherical shape in which the diameter in the direction perpendicular to the insertion direction from insertion port 8 is set slightly larger than the diameter in the insertion direction from insertion port 8. Thereby, a hand can be easily put on placement surface 14.

As illustrated in FIG. 5, light incident port 16 is provided on the lower end surface of light guide body 12. Light source 17 is disposed at incident port 16. Further, as shown in FIG. 6, light guide body 12 is provided with a plurality of incident ports 16, and light sources 17 corresponding to respective incident ports 16. Light guide body 12 is configured to enable the light from light source 17 to enter the light guide path via incident port 16. The light irradiated from light source 17 has a main wavelength range of near-infrared light (about 750 nm to about 2 μm) which is preferred as light for therapeutic treatment of articular rheumatism.

Light guide body 12 may be made of a material having a light-guiding property with respect to the therapeutic light from light source 17. In Embodiment 1, a case is described in which light guide body 12 is made of a material having a refractive index higher than a refractive index of a biological tissue. The refractive index of a biological tissue is different depending on the part and state of the biological tissue, but is generally in the range of 1.4 to 1.5. In Embodiment 1, the refractive index of a biological tissue is assumed to be 1.4, and the refractive index of the material of light guide body 12 is set higher than 1.4.

Examples of the material of light guide body 12 include transparent acrylic resin (refractive index: 1.49), transparent polyethylene terephthalate (PET) resin (refractive index: 1.66), transparent glass (refractive index: 1.51), transparent silicone resin having a refractive index adjusted to be higher than 1.4, and the like.

In phototherapy apparatus 1 configured as described above, light emitted from light source 17 is made to enter the inside of light guide body 12. The light having entered the inside first reaches the inner surface of placement surface 14 or the inner surface of back-facing surface 15. The light having reached the inner surface of placement surface 14 or of back-facing surface 15 is emitted to the outside or reflected on the inner surface according to the incident angle of the light with respect to the inner surface. That is, when the incident angle of the light with respect to the inner surface is smaller than the critical angle derived from the material of light guide body 12 and the outside environment (assumed to be air at this time) at the part of the inner surface reached by the light, the light is emitted to the outside of light guide body 12. On the other hand, when the incident angle of the light with respect to the inner surface is larger than the critical angle, the light is reflected on the inner surface.

Since the thickness of the light guide path is substantially uniform, the light reflected on the inner surface propagates in the light guide path while repeating reflection at substantially the same incident angle with respect to the inner surface. The light propagating in the light guide path while repeating reflection is principally trapped inside light guide body 12 when nothing is brought into contact with light guide body 12 (that is, when air is in contact with light guide body 12).

On the other hand, when a substance (e.g., a biological tissue, such as a hand) having a refractive index higher than the refractive index (1.0) of air is brought into contact with placement surface 14, the light, which is not emitted to the outside at the time when air is in contact with placement surface 14, is emitted to the outside. That is, when a substance having a refractive index higher than the refractive index (1.0) of air is brought into contact with placement surface 14, the critical angle on the boundary surface of the contact portion is increased. The portion of the light corresponding to an increase in the critical angle is emitted to the outside of light guide body 12 from the contact portion.

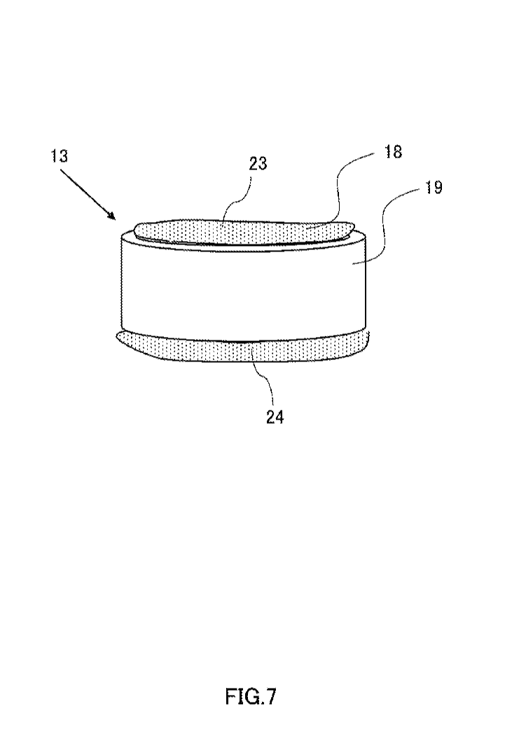

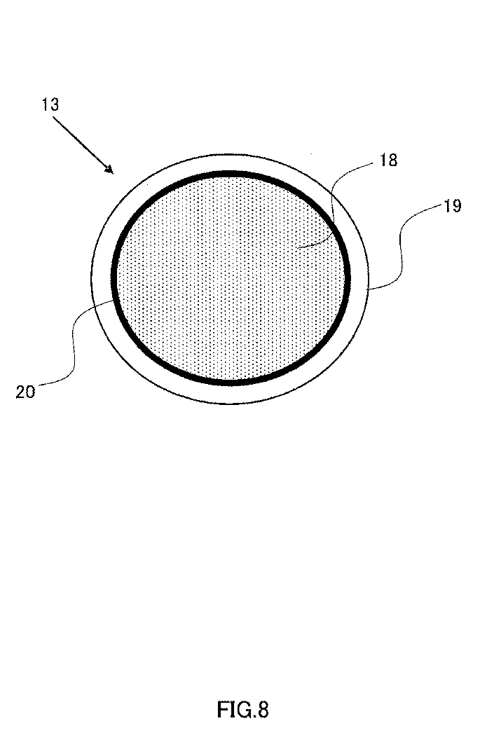

Next, auxiliary light guide body 13 is described with reference to FIGS. 7 and 8. FIG. 7 is a perspective view of auxiliary light guide body 13. FIG. 8 is a top view of auxiliary light guide body 13. Auxiliary light guide body 13 is configured by hollow holding member 19 and light guide member 18. Auxiliary light guide body 13 is configured by inserting and holding light guide member 18 in the hollow part of holding member 19.

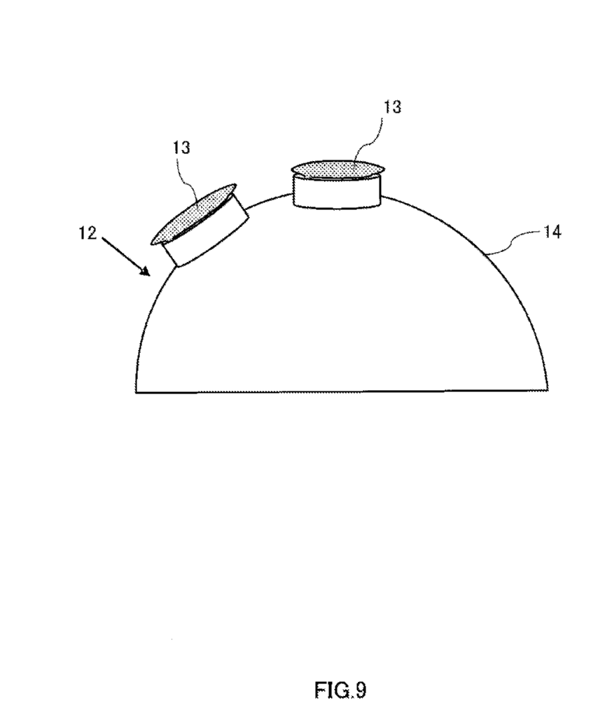

As shown in FIG. 9, auxiliary light guide body 13 is disposed at a part of placement surface 14 of light guide body 12 so as to be in contact with light guide member 18. Light guide member 18 can be disposed at any place of placement surface 14 of light guide body 12, and is also configured to be detachable.

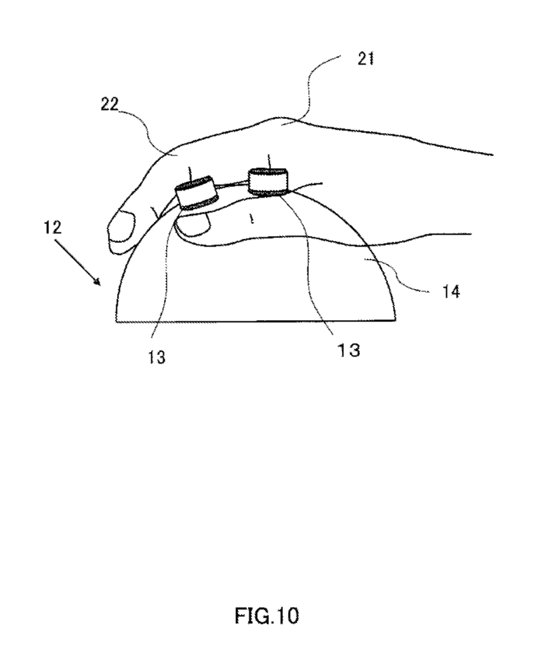

As shown in FIG. 10, therapeutic treatment is performed in such a manner that affected part, such as a hand and fingers, is placed on placement surface 14, and that a treatment part, such as finger joints, is placed in contact with light guide member 18 of auxiliary light guide body 13. That is auxiliary light guide body 13 is used in the state of being inserted between the treatment part and light guide body 12.

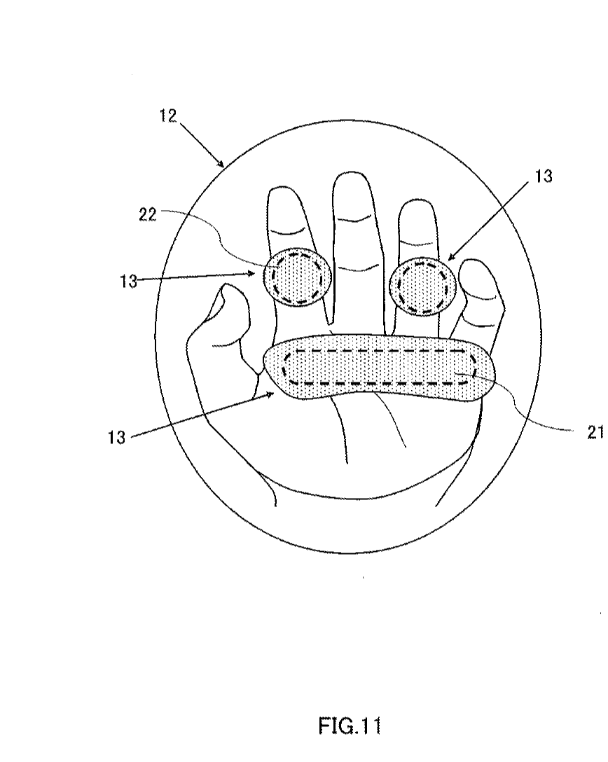

Embodiment 1 is described by taking, as an example, phototherapeutic treatment of a hand or fingers of a patient with rheumatism. The affected parts of a hand or fingers of a patient with rheumatism in which the severest symptoms appear are metacarpophalangeal joint 21 and proximal interphalangeal joint 22. Auxiliary light guide body 13 inserted between light guide body 12 and each of metacarpophalangeal joint 21 and proximal interphalangeal joint 22 as treatment parts is placed as shown in FIG. 11. Light guide member 18 of auxiliary light guide body 13 is mainly brought into contact with light guide body 12.

Next, light guide member 18 of auxiliary light guide body 13 is described. Light guide member 18 is made of a material having the following three features.

The first feature is that the material has a light-guiding property. This is because, as will be described below, in the phototherapy apparatus according to the present invention, therapeutic light in light guide body 12 is applied to a treatment part via light guide member 18. The material of light guide member 18 is not limited in particular as long as it has a light-guiding property. In Embodiment 1, a material having a relationship of refractive indexes expressed by Equation 1 is used as an example of the material of the light-guiding property.

[1]

Refractive index of air<Refractive index of biological tissue<Refractive index of light guide body<Refractive index of light guide member (Equation 1)

The second feature is that the material has at least softness, such as flexibility and resilience. As described above, auxiliary light guide body 13 is used in the state of being inserted between the affected part and placement surface 14. When light guide member 18 does not have softness, light guide member 18 is difficult to be brought into close contact with the treatment part which is a bent part, such as a finger joint. When light guide member 18 and the treatment part are not in close contact with each other, sufficient light cannot be applied to the treatment part. When light guide member 18 has such softness that the shape thereof is changed according to the bent part of the treatment part, light guide member 18 and the affected part can be brought into close contact with each other. Further, when light guide member 18 has softness, light guide member 18 can be disposed in close contact with placement surface 14 of each of light guide bodies 12 having various shapes (placement surface 14 in Embodiment 1 is a substantially semi-spherical surface).

The third feature is that the material has surface tackiness (adhesiveness). That is, light guide member 18 is disposed on placement surface 14 of light guide body 12, and is brought into close contact with placement surface 14 by applying slight pressing force, or the like, to light guide member 18. Thereby, light guide member 18 and placement surface 14 can substantially have no gap therebetween. For this reason, it is preferred that the material of light guide member 18 has a close contact property with a material constituting light guide body 12 (e.g., transparent acrylic resin, transparent polyethylene terephthalate (PET) resin, transparent glass, and transparent silicone resin having a refractive index adjusted to be higher than 1.4).

In the case where light guide member 18 has surface tackiness, the placement position of auxiliary light guide body 13 can be fixed when auxiliary light guide body 13 is merely disposed on placement surface 14 of light guide body 12, or when auxiliary light guide body 13 is only slightly pressed after being disposed on placement surface 14. Further, auxiliary light guide body 13 needs not be fixed by a tape, or the like, and auxiliary light guide body 13 can also be easily removed.

In this way, even when phototherapeutic treatment (photo-irradiation) is performed by inserting auxiliary light guide body 13 between a treatment part and placement surface 14, the position of auxiliary light guide body 13 initially disposed on placement surface 14 is hardly shifted because of the surface tackiness of auxiliary light guide body 13.

An elastomer having a light-guiding property is an example of the materials having the above-described three features and having the relationship between the refractive indexes expressed by Equation 1. Specifically, when the material of light guide body 12 is assumed to be transparent acrylic resin, transparent polyethylene terephthalate (PET) resin, or transparent glass, examples of elastomers having the three features include transparent silicone resin having a refractive index adjusted to be higher than the refractive index of the material of light guide body 12, isoprene rubber (refractive index: 1.52), ethylene propylene rubber (refractive index: 1.48), chloroprene rubber (refractive index: 1.558), butyl rubber refractive index: 1.508), urethane rubber (refractive index: 1.50 to 1.55), epichlorohydrin rubber (refractive index: 1.51), butadiene rubber (refractive index: 1.516), styrene butadiene rubber (refractive index: 1.535), nitrile rubber (refractive index: 1.52), and the like.

When transparent silicone resin is used as the material of each of light guide body 12 and light guide member 18, it is necessary that the refractive index of the transparent silicone resin of light guide member 18 is adjusted to be higher than the refractive index of transparent silicone resin of light guide body 12. Further, when transparent silicone resin is used as the material of light guide body 12, and when isopropylene rubber is used as the material of light guide member 18, it is necessary that the refractive index of transparent silicone resin is adjusted to be lower than the refractive index (1.52) of isopropylene rubber.

As described above, holding member 19 has a shape having a hollow part therein. Light guide member 18 is configured to be inserted and held in the hollow part of holding member 19. In Embodiment 1, holding member 19 is cylindrical in shape.

It is preferred that holding member 19 be made of a rigid material, such as plastic, which is not optically transparent. Auxiliary light guide body 13 may not have holding member 19. However, when light is applied to a treatment part via auxiliary light guide body 13, holding member 19 prevents the light from leaking from a portion not in contact with the treatment part.

Reflection film 20 may be provided on the inner surface of the hollow part of holding member 19. When light is applied to an affected part from light guide body 12 via light guide member 18, reflection film 20 enables the light to be efficiently applied to the affected part while preventing the light from leaking from a portion of light guide member 18, which portion is not in contact with the treatment part.

In auxiliary light guide body 13 in Embodiment 1, light guide member 18 held in the hollow part of holding member 19 is raised from the peripheral edge of the hollow part of holding member 19 as shown in FIG. 7. For example, the upper side of auxiliary light guide body 13 of FIG. 7 is assumed to be treatment part contact surface 23 to be brought into contact with a treatment part. When the portion of treatment part contact surface 23 of light guide member 18 is raised and held, light guide member 18 can be brought into close contact with a finger joint which is a bent part. This is because the shape of light guide member 18 can be changed so as to fit the bent portion of the joint. On the other hand, when light guide member 18 is held in a state of not being raised from the peripheral edge of the hollow part of holding member 19 (a state where light guide member 18 is held inside the peripheral edge of the hollow part of holding member 19), the finger joint is hardly brought into close contact with light guide member 18. This is because holding member 19 is made of a rigid material and the finger joint is a bent part.

On the other hand, the underside of auxiliary light guide body 13 of FIG. 7 is used as holding surface 24 which is brought into contact with placement surface 14. When light guide member 18 is also raised on holding surface 24, the positions of light guide body 12 and auxiliary light guide body 13 can be fixed to some extent so as not to be displaced from each other. Thereby, the positional displacement of auxiliary light guide body 13 is prevented at the time when the affected part is placed on auxiliary light guide body 13 for receiving therapeutic treatment using auxiliary light guide body 13.

When light guide member 18 is also raised on holding surface 24, the light from light guide body 12 can also be made to suitably enter light guide member 18. That is, when a gap exists between light guide body 12 and light guide member 18, the boundary surface between the gap portion and light guide body 12 substantially becomes a boundary surface between light guide body 12 and air, and hence the light is difficult to be emitted from light guide body 12. When a raised portion of light guide member 18 is provided on holding surface 24 of auxiliary light guide body 13 of FIG. 7, auxiliary light guide body 13 can be disposed in the state where the close contact state between light guide member 18 and placement surface 14 is improved.

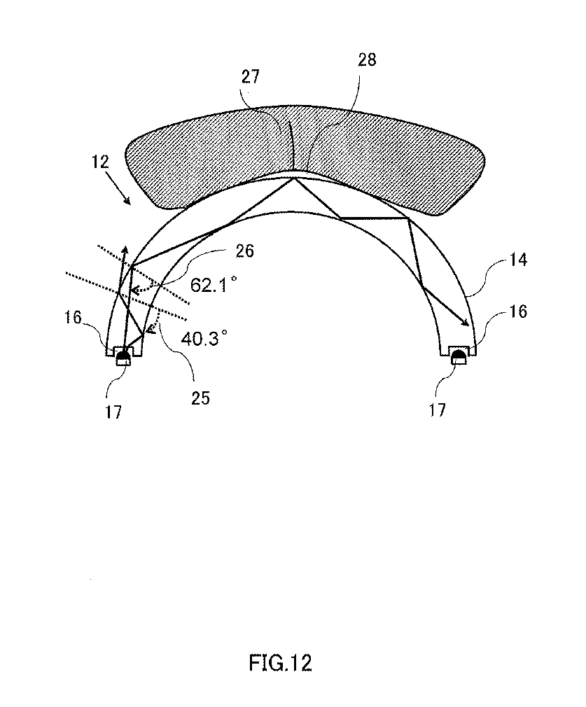

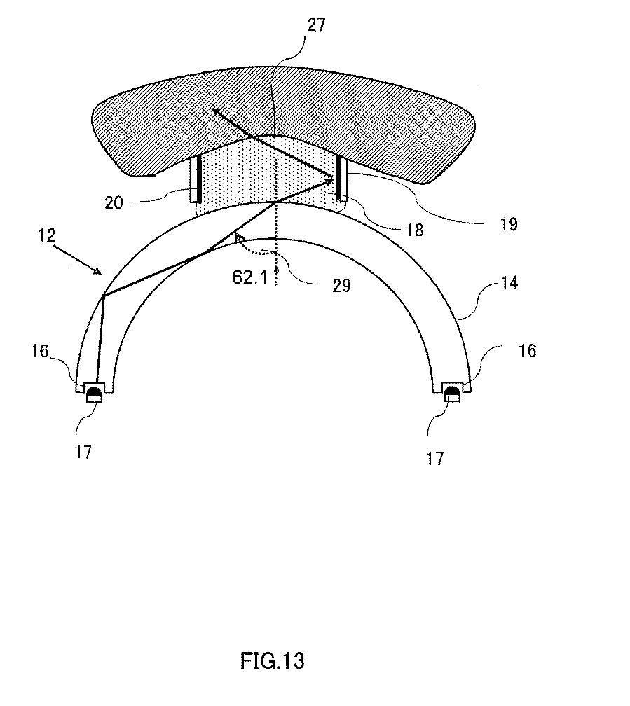

The state where light is irradiated during the phototherapeutic treatment using auxiliary light guide body 13 configured as described above is described with reference to FIGS. 12 and 13. In FIG. 12, a case is described in which acrylic resin (refractive index: 1.49) is used as the material of light guide body 12, and light guide member 18 is not disposed. In FIG. 13, a case is described in which an auxiliary light guide body including light guide member 18 made of isoprene rubber (refractive index: 1.52) is disposed.

FIG. 12 is a view illustrating a light irradiation state in the case where light guide member 18 is not disposed. That is, FIG. 12 is a view illustrating a case where conventional phototherapeutic treatment is performed. In FIG. 12, when a treatment part is not brought into contact with light guide body 12, placement surface 14 of light guide body 12 is in contact with air. When light is emitted from acrylic resin (refractive index: 1.49) as the material of light guide body 12 into air (refractive index: 1.00), the critical angle of the light at the boundary surface is 42.155 degrees. For this reason, as shown in FIG. 12, light having reached the inner wall surface of light guide body 12 at an incident angle of 42.155 degrees or more in light guide body 12 is totally reflected on the inner wall surface. The reflected light repeats reflection on the inner wall surface of light guide body 12. On the other hand, light having reached the inner wall surface of light guide body 12 at an incident angle smaller than 42.155 degrees in light guide body 12 is emitted to the outside from light guide body 12.

That is, FIG. 12, light having reached the inner wall surface of placement surface 14 at an angle of 40.3 degrees (incident angle a: 25) has an incident angle smaller than the critical angle (42.155 degrees), and hence is emitted to the outside from placement surface 14. On the other band, light having reached the inner wall surface of placement surface 14 at an angle of 62.1 degrees (incident angle b: 26) has an incident angle larger than the critical angle (42.155 degrees), and hence is totally reflected.

A portion of light guide body 12 having a uniform thickness in which light is emitted to the outside is substantially limited only to the vicinity of incident port 16 of light source 17. This is because the portion where light having a large incident angle reaches placement surface 14 is limited only to the vicinity of incident port 16 of light source 17. As a result, in the state where air is in contact with placement surface 14 of light guide body 12, the amount of light emitted to the outside from placement surface 14 is very small.

When a hand and fingers are placed on placement surface 14 of light guide body 12, a part of placement surface 14 and biological tissue as the hand and fingers are brought into close contact with each other. When light is emitted to the biological tissue (refractive index: 1.40) from acrylic resin (refractive index: 1.49) used as the material of light guide body 12, the light has a critical angle of 69.984 degrees at the boundary surface. For this reason, light having reached the inner surface of placement surface 14 at an angle smaller than 69.984 degrees is emitted from placement surface 14.

Among affected parts of a hand and fingers of a patient with rheumatoid arthritis, the affected part which most needs to be treated is finger joint 27. However, finger joint 27 is a bent part, and hence gap 28 is easily generated between the bent part and placement surface 14 of light guide body 12. A healthy person and a patient with slight symptoms of rheumatism can prevent the generation of gap 28 by bringing the bent part into close contact with light guide body 12 according to the shape of light guide body 12. However, it is difficult for a patient with severe rheumatism accompanied by joint deformation to prevent the generation of gap 28 by bringing the bent part into close contact with light guide body 12. As a result, sufficient light is not applied to finger joint 27 which most needs to receive the therapeutic treatment.

On the other hand, FIG. 13 is a vie illustrating a light irradiation state in the case where auxiliary light guide body 13 is disposed on light guide body 12. That is, FIG. 13 is a view illustrating a case where phototherapeutic treatment is performed by using the phototherapy apparatus according to the present invention. As shown in FIG. 13, when auxiliary light guide body 13 is disposed on placement surface 14 of light guide body 12, light guide member 18 is brought into close contact with placement surface 14. This is because light guide member 18 has a close contact property and softness, and also light guide member 18 is held and raised from the peripheral edge of the hollow part of holding member 19. That is, almost no gap is generated between light guide body 12 and light guide member 18. Therefore, placement surface 14 of light guide body 12 and the isoprene rubber serving as light guide member 18 are brought into close contact with each other.

The refractive index (1.52) of isoprene rubber is higher than the refractive index (1.49) of acrylic resin, and hence all the light having reached placement surface 14 of light guide body 12 enters light guide member 18 from light guide body 12 as shown in FIG. 13. That is, as can be seen from FIG. 13, even the light having the incident angle of 62.1 degrees (incident angle c: 29) is emitted from placement surface 14. As is apparent from the comparison of FIG. 13 with FIG. 12, the amount of light emitted from placement surface 14 of light guide body 12 in the vicinity of finger joint 27 is increased.

The light having entered light guide member 18 propagates toward finger joint 27. When the light enters light guide member 18, the light propagating in the direction different from the direction to finger joint 27 is reflected by reflection film 20 provided, on the inner surface of holding member 19, so as to be guided in the direction toward finger joint 27.

Since light guide member 18 has a light-guiding property, softness, and surface tackiness, and since light guide member 18 is held so as to be raised from the peripheral edge of the hollow part of holding member 19, light guide member 18 is brought into close contact with finger joint 27 without a gap. That is, substantially no gap exists between light guide member 18 and finger joint 27. The refractive index (1.52) of the isoprene rubber as the material of light guide member 18 is higher than the refractive index (1.40) of the biological tissue. Since the critical angle at the boundary surface between light guide member 18 and finger joint 27 is 67.08 degrees, the light having entered the boundary surface at an angle of 67.08 degrees or more is totally reflected, and a part of the light enters the biological tissue after being repeatedly reflected by reflection film 20, and the like, of the inner wall of holding member 19.

With the above-described configuration, even in therapeutic treatment of a patient with serious rheumatism accompanied by joint deformation, sufficient therapeutic light can be supplied to a deep part of tissue of an affected part without requiring an unnatural action of the patient and without giving pin and suffering to the patient. As a result, suitable therapeutic treatment can be performed.

Further, when auxiliary light guide body 13 is disposed on a part of placement surface 14 of light guide body 12, more intense light can be applied to the affected part. Specifically, when a hand and fingers as affected parts are directly placed on the placement surface of light guide body 12 without disposing auxiliary light guide body 13 on placement surface 14 of light guide body 12, the whole of the hand and fingers are brought into contact with placement surface 14 of light guide body 12. Therefore, the contact area of placement surface 14 with the hand and fingers is increased. Therefore, light is applied to a part to which the light needs not be applied. However, the amount of therapeutic light from light source 17 is limited. Therefore, when the contact area of placement surface 14 with the hand and fingers is too large, the amount of the therapeutic light supplied to the treatment part is relatively reduced, and hence sufficient therapeutic light cannot be applied to the treatment part.

In the present invention, the main portion in contact with placement surface 14 can be limited to light guide member 18 by using auxiliary light guide body 13, and thereby the therapeutic light from light guide body 12 can be collected to auxiliary light guide body 13 disposed on placement surface 14. As a result, the therapeutic light applied to the treatment part can be increased.

### Embodiment 2

Embodiment 2 as a modification of Embodiment 1 describes a configuration in which the relationship in the refractive index between light guide body 12 and light guide member 18 of auxiliary light guide body 13 is set as expressed by Equation 2. The other parts of the configuration are the same as those in Embodiment 1, and hence explanation thereof is omitted.

[2]

Refractive index of air<Refractive index of light guide body<Refractive index of light guide member≦Refractive index of biological tissue (Equation 2)

The material of light guide body 12 is a material which is transparent to the light from light source 17, and which has a refractive index higher than the refractive index (1.0) of air and lower than the refractive index of the biological tissue. Examples of such material include perfluoro resin, silicone resin having a refractive index adjusted to 1.4 or less, FEP (tetrafluoroethylene-hexafluoropropylene copolymer) resin, polytetrafluoroethylene, and the like.

Examples of the material of light guide member 18, which satisfies Equation 2, include elastomers having a light-guiding property. Examples of such elastomers include fluororubber (refractive index: 1.38), and silicone resin having a refractive index adjusted to 1.4 or less. When the refractive index of the biological tissue is 1.5, the elastomer may be ethylene propylene rubber (refractive index: 1.48), acrylic rubber (refractive index: 1.465), and the like.

When silicone resin having a refractive index adjusted to 1.4 or less is used as the material of light guide body 12, the refractive index of the silicone resin must be adjusted to a value equal to or lower than the refractive index of light guide member 18. Further, when silicone resin having a refractive index adjusted to 1.4 or less is used as the material of light guide body 12 and light guide member 18, the refractive index of each of light guide body 12 and light guide member 18 needs to be adjusted to satisfy Equation 2.

A state of light irradiation in the phototherapeutic treatment using auxiliary light guide body 13 configured as described above is described with reference to FIG. 13, in an example in which perfluoro resin (refractive index: 1.34) is used as the material of light guide body 12 and in which silicone resin having a refractive index adjusted to 1.4 is used as the material of light guide member 18. As shown in FIG. 13, when auxiliary light guide body 13 is used, the boundary surface between light guide member 18 and placement surface 14 is formed by perfluoro resin and silicone resin.

Light having entered the light guide path of light guide body 12 from light source 17 first reaches the inner surface of placement surface 14 or the inner surface of back-facing surface 15. When the light having reached the inner surface of placement surface 14 or of back-facing surface 15, and when the incident angle of the light with respect to the inner surface is smaller than the critical angle derived from the material of light guide body 12 and air (refractive index: 1.0), the light is emitted to the outside from light guide body 12.

On the other hand, when the incident angle of the light having readied the inner surface is larger than the critical angle, the light is reflected on the inner surface. Then, the reflected light propagates while repeating the reflection. At this time, the light guide path has a substantially uniform thickness, and hence the incident angle of the light with respect to the inner surface is substantially fixed.

When the light propagating while repeating the reflection reaches a part on which light guide member 18 is disposed within placement surface 14 of light guide body 12, all the light enters light guide member 18 in principle without causing total reflection. This is because the refractive index (1.4) of silicone resin used as the material of light guide member 18 is higher than the refractive index (1.34) of perfluoro resin.

The light having entered light guide member 18 reaches the boundary surface between light guide member 18 and finger joint 27 after reflection on reflection film 20 and the like. The refractive index of light guide member 18 is equal to or lower than the refractive index of the biological tissue, and hence all the light also enters finger joint 27 in principle without being totally reflected by the boundary surface.

With the above-described configuration, all the light having reached the boundary surface between light guide member 18 and the affected part from light guide body 12 through light guide member 18 can be made, in principle, to enter the affected part from light guide member 18. Thereby, light can be supplied to a bent part, such as finger joint 27 without requiring an unnatural action of a patient with rheumatoid arthritis and without giving pain and suffering to the patient. Further, even when the portion of finger joint 27 in particular needs to be intensively treated, light can be highly efficiently supplied to the finger joint portion, and hence the improvement of the therapeutic efficacy can be expected.

### Embodiment 3

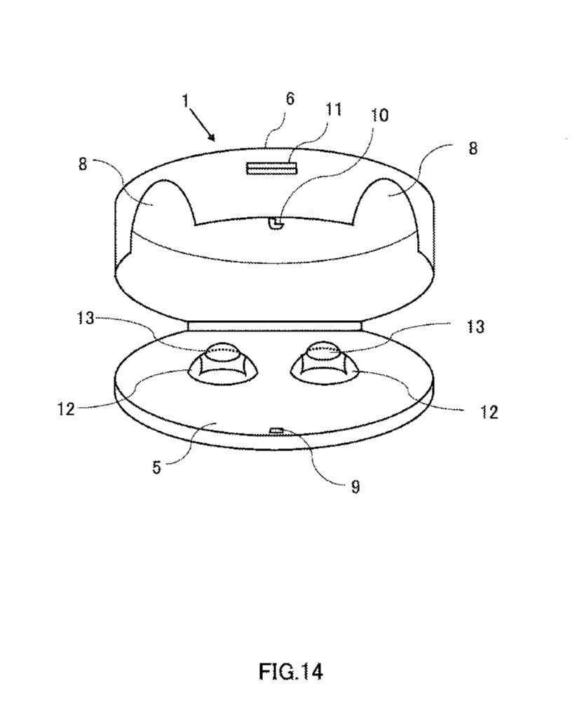

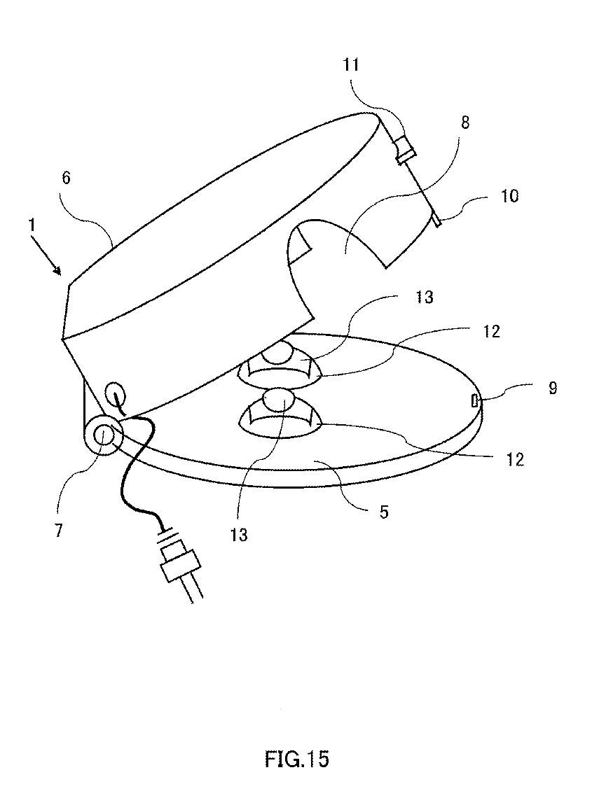

As shown in FIGS. 14 and 15, Embodiment 3 is different in the configuration of auxiliary light guide body 13 from Embodiment 1 and Embodiment 2. The other parts of the configuration are the same as those in Embodiment 1 and Embodiment 2, and hence explanation thereof is omitted. Further, the materials of light guide body 12 and light guide member 18 are not particularly limited as long as the materials have light-guiding properties. However, in Embodiment 3, a case is described in which the refractive indexes of the materials have the same relationship (see Equation 2) as that in Embodiment 2.

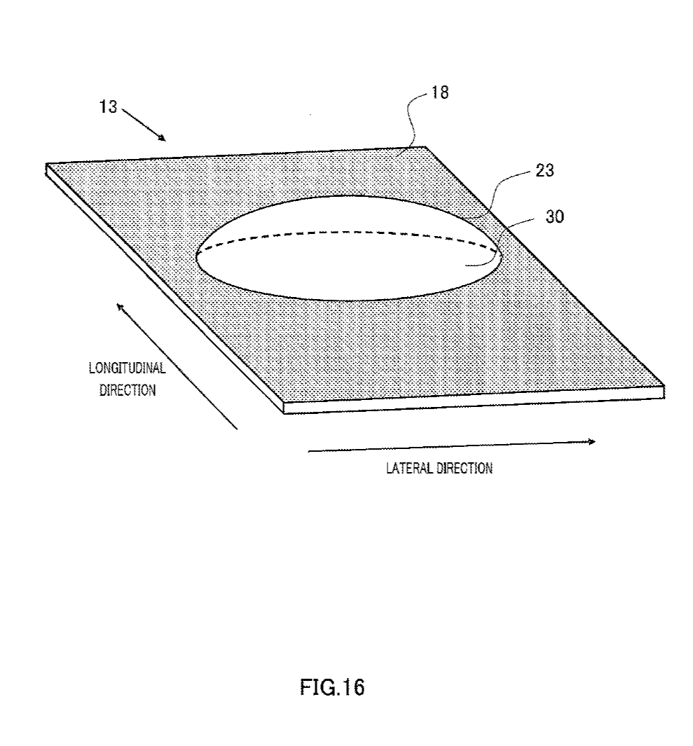

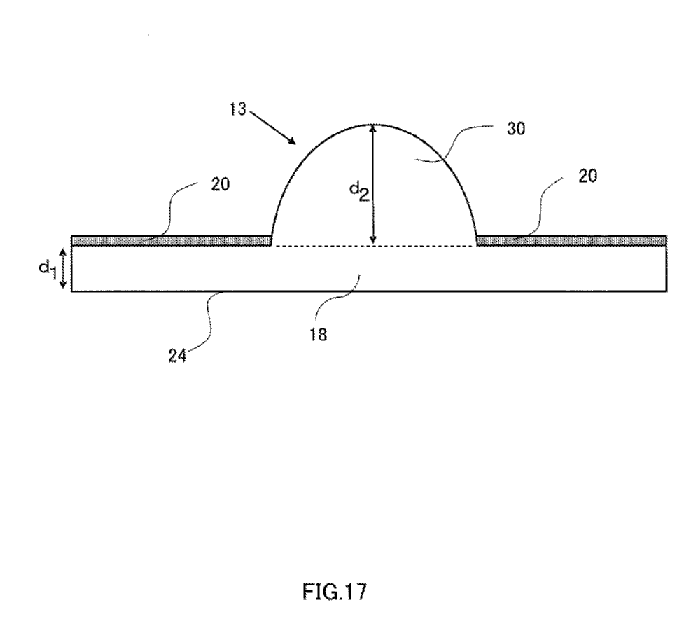

Auxiliary light guide body 13 in Embodiment 3 is illustrated in FIGS. 16 and 17. FIG. 16 is a perspective view of auxiliary light guide body 13. FIG. 17 is a longitudinal cross-sectional view of auxiliary light guide body 13.

Auxiliary light guide body 13 is configured by light guide member 18 and reflection film 20. Light guide member 18 is substantially sheet-shaped. The surface of light guide member 18 is the surface on which an affected part is placed. The back surface of light guide member 18 is holding surface 24 which is disposed on placement surface 14 of light guide body 12.

A part of the surface of light guide member 18 is treatment part contact surface 23 which is brought into contact with a treatment part. In treatment part contact surface 23, the thickness of light guide member 18 is set larger than the other parts thereof. That is, treatment part contact surface 23 is formed as projecting section 30. As shown in FIG. 16, projecting section 30 has the lateral width larger than the longitudinal width. Treatment part contact surface 23 needs only to have a size sufficient for placing an affected part thereon. Further, as will be described below, auxiliary light guide body 13 is used by being disposed on placement surface 14 of light guide body 12, and hence has a size smaller than the size of placement surface 14.

Reflection film 20 is provided on the surface of light guide member 18 except projecting section 30. It is only necessary that reflection film 20 is configured to reflect the light in light guide member 18 from the surface side in the direction toward holding surface 24. For example, the surface of light guide member 18 is coated with silver color, gold color, or the like except projecting section 30.

Similarly to Embodiment 1 and Embodiment 2, the material of light guide member 18 has surface tackiness, softness, and a light-guiding property, and may be an elastomer or the like.

In the following, a propagation state is described in which light is supplied to a treatment part from light source 17 via light guide body 12 and light guide member 18. The refractive indexes of the respective sections are not limited in particular, and the respective sections may be configured such that the light from light source 17 is supplied to the treatment part via light guide body 12 and light guide member 18. However, in Embodiment 3, the relationship between, the refractive index of light guide body 12 and the refractive index of light guide member 18 is set as expressed by Equation 3.

[3]

Refractive index of air<Refractive index of light guide body<Refractive index of light guide member≦Refractive index of biological tissue (Equation 3)

Refractive index of a biological tissue is different depending on the part and state of the biological tissue, but is roughly in the range of 1.4 to 1.5. In Embodiment 3, the refractive index of the biological tissue is set to 1.4. Specific examples of the material of light guide body 12, which material satisfies Equation 3, includes perfluoro resin (refractive index: 1.34), FEP resin (refractive index: 1.34), polytetrafluoroethylene (refractive index: 1.35), and silicone resin having a refractive index adjusted to a value in the range expressed by Equation 3. Further, specific examples of the material of light guide member 18 includes fluororubber (refractive index: 1.38), and silicone resin having a refractive index adjusted to a value in the range expressed by Equation 3. The materials of light guide body 12 and light guide member 18 are not limited to these.

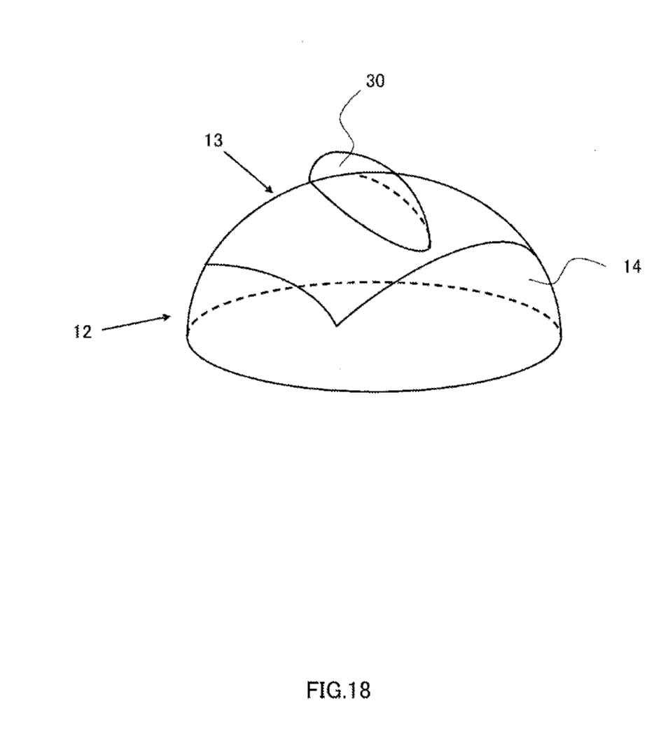

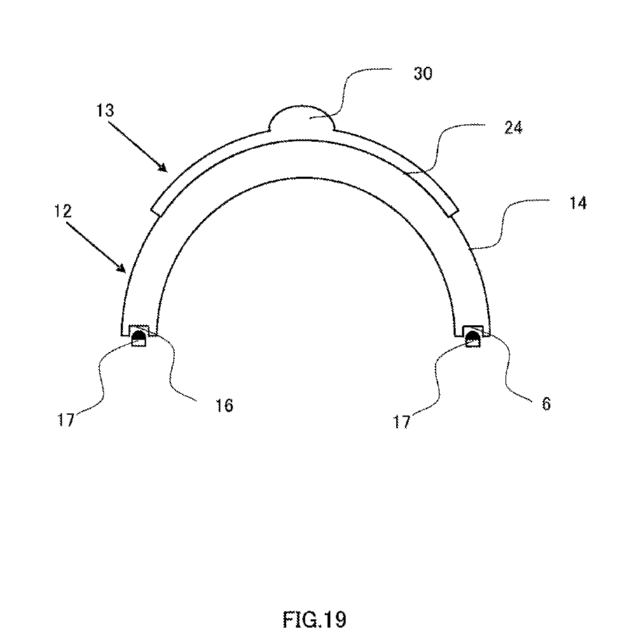

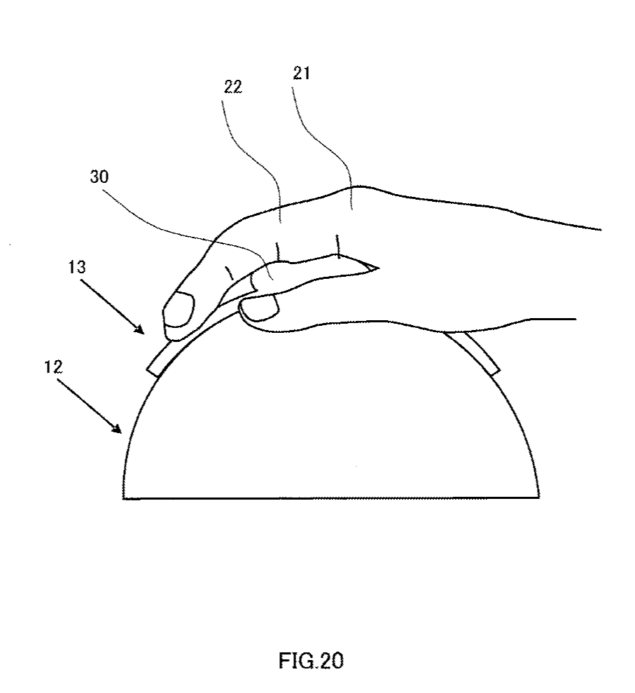

Auxiliary light guide body 13 configured as described above is disposed on placement surface 14 of light guide body 12 as illustrated in FIGS. 18 and 19. Then, as illustrated in FIG. 20, phototherapeutic treatment is performed by placing an affected part so that a treatment part is brought into contact with projecting section 30. In Embodiment 3, an example is described in which a finger joint of a hand of a patient with rheumatoid arthritis is treated. In the hand of a patient rheumatoid arthritis, the parts in which symptoms most notably appear are metacarpophalangeal joint 21 and proximal interphalangeal joint 22. Therefore, the treatment parts in Embodiment 3 mainly mean metacarpophalangeal joint 21 and proximal interphalangeal joint 22.

Since light guide member 18 of auxiliary light guide body 13 has softness, auxiliary light guide body 13 can be disposed on placement surface 14 of light guide body 12 so that holding surface 24 of auxiliary light guide body 13 matches the substantially semi-spherical shape of placement surface 14 of light guide body 12. Further, when bent parts, such as metacarpophalangeal joint 21 and proximal interphalangeal joint 22, which are treatment parts, are placed on projecting section 30, the shape of projecting section 30 is changed due to the softness of light guide member 18. Thereby, projecting section 30 can be brought into close contact with the bent parts.

The degree of softness of light guide member 18 is suitably set according to use, and needs only to be set to such an extent that, when the treatment part is placed on projecting section 30 of light guide member 18, the shape of projecting section 30 is changed according to the shape of the treatment part so as to enable projecting section 30 and the treatment part to be brought into close contact with each other. That is, light guide member 18 may have a level of softness such that, when a hand is placed on the affected part placement surface of light guide member 18, the shape of projecting section 30 is changed by the weight of the hand, or pressure such as slight pressing pressure. For example, the level of softness corresponds to about 3° to 30° in terms of rubber hardness. Note that it is preferred that light guide member 18 used in Embodiment 1 and Embodiment 2 also has the same level of hardness.

Since light guide member 18 has surface tackiness (adhesiveness), the placement position of auxiliary light guide body 13 can be fixed only by disposing auxiliary light guide body 13 on placement surface 14 of light guide body 12, or only by slightly pressing auxiliary light guide body 13 after auxiliary light guide body 13 is disposed thereon. Since it is also not necessary to fix light guide member 18 by a tape, or the like, auxiliary light guide body 13 can be detached only by peeling off auxiliary light guide body 13, in this way, auxiliary light guide body 13 can be easily attached and detached. Further, even when an affected part is placed on the surface of light guide member 18 in order to receive phototherapeutic treatment, the placement position of auxiliary light guide body 13 on placement surface 14 of light guide body 12 is not shifted.

Auxiliary light guide body 13 is held on placement surface 14 of light guide body 12 by holding surface 24 of auxiliary light guide body 13. An affected part is placed on the surface of projecting section 30 of auxiliary light guide body 13. For this reason, several mm of thickness d₁ (distance between holding surface 24 and the surface (see FIG. 17)) of light guide member 18 of auxiliary light guide body 13 is sufficient, and it is not necessary to increase thickness d₁ more.

Further, the shape and thickness (height from the surface of light guide member 18 to the top of projecting section 30) of projecting section 30 can be suitably set according to the affected part to be treated. For example, when a finger joint is to be treated, projecting section 30 needs to have a thickness of at least about 1 cm in order to be brought into close contact with the finger joint.

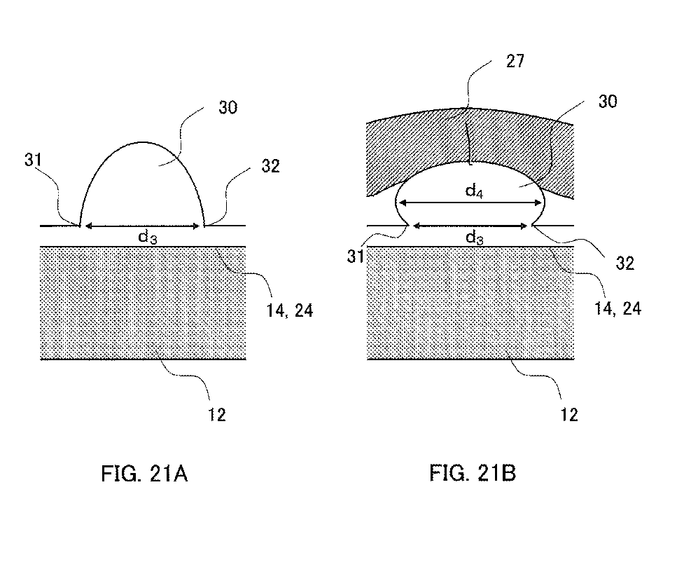

Next, the reason why projecting section 30 is brought into close contact with a treatment part which is a bent part such as a finger joint, when the treatment part is placed on projecting section 30, is described with reference to FIG. 21. FIG. 21A is a view illustrating a state where finger joint 27 is not placed on projecting section 30. FIG. 21B is a view illustrating a state where finger joint 27 is placed on projecting section 30. In FIG. 21A and FIG. 21B, the configuration of reflection film 20 is omitted in order to facilitate explanation.

As shown in FIG. 21A, when finger joint 27 is not placed on projecting section 30, substantially semi-spherical projecting section 30 is formed within distance d₃. Distance d₃ is the width of projecting section 30 projecting from light guide member 18, and the distance between boundary 31 and boundary 32.

Light guide member 18 has softness, and hence when finger joint 27 is placed on projecting section 30, projecting section 30 is deformed, as shown in FIG. 21B, in the lateral and longitudinal directions by application of pressure from the placed hand. Specifically, the size of the most deformed portion becomes larger than distance d₃. Finger joint 27 and the vicinity thereof are brought into contact with deformed projecting section 30.

Therefore, even when the placement position of the top of projecting section 30 is slightly deviated from the position of finger joint 27, finger joint 27 and the vicinity thereof can be brought into contact with projecting section 30 by shape change of projecting section 30. For this reason, when a hand is placed on the surface of light guide member 18, it is not necessary that finger joint 27 of the band and projecting section 30 of light guide member 18 are strictly aligned with each other. That is, efficient phototherapeutic treatment can be performed only by simply placing a hand on the surface of light guide member 18.

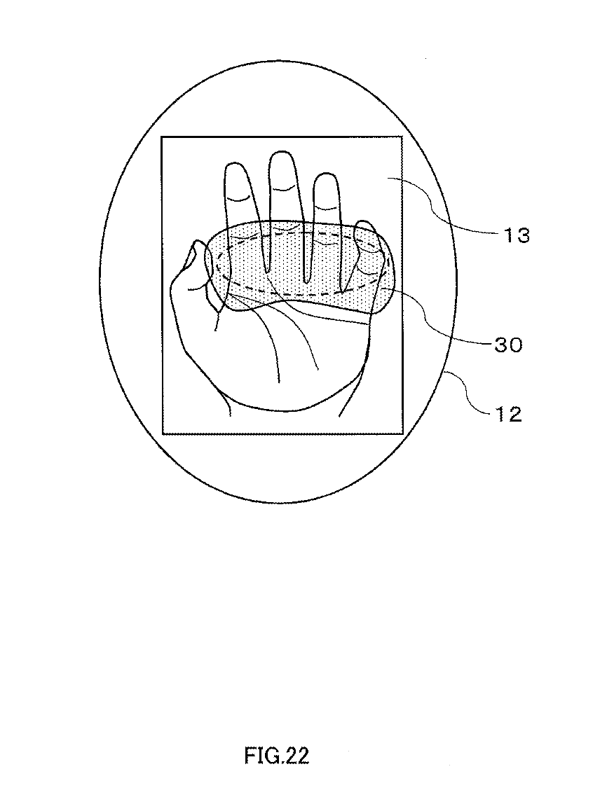

In FIG. 21A and FIG. 21B, a configuration, in which one projecting section 30 is disposed in correspondence with one finger joint, is illustrated, but the configuration is not limited to this. As shown in FIG. 22, integrated projecting section 30 configured to be able to be brought into close contact with both metacarpophalangeal joint 21 and proximal interphalangeal joint 22 (see FIG. 20) may also be used. The broken line of projecting section 30 in FIG. 22 indicates the position of projecting section 30 before the affected part is placed thereon. The solid line of projecting section 30 in FIG. 22 indicates the position of projecting section 30 whose shape is changed by placing the affected part thereon. Deformed projecting section 30 is brought into contact with the whole of metacarpophalangeal joint 21 and the whole of proximal interphalangeal joint 22.

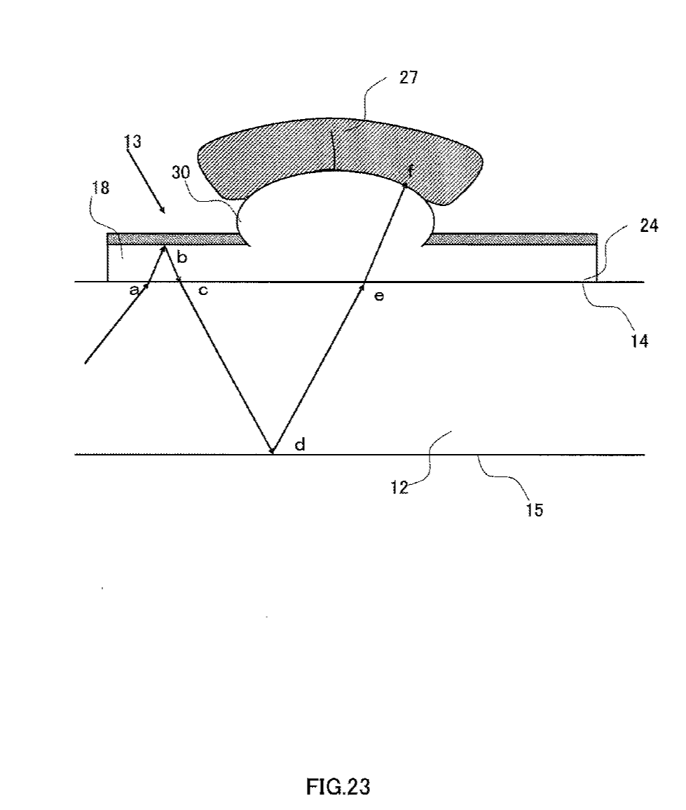

FIG. 23 illustrates an example in the state where light is applied to an affected part by using phototherapy apparatus 1 of Embodiment 3. Light, having entered the inside of light guide body 12 having a substantially uniform thickness from the light source, propagates between the inner surface of placement surface 14 and the inner surface of back-facing surface 15 of light guide body 12 while repeating reflection and maintaining a substantially fixed reflection angle. When the light, propagating in light guide body 12 reaches the inner surface (a in FIG. 23) of placement surface 14, on which auxiliary light guide body 13 is disposed, all the light enters light guide member 18 in principle without being totally reflected. This is because the boundary surface of the part reached by light (“a” in FIG. 23) is formed by light guide body 12 and light guide member 18, and the refractive index of light guide member 18 is higher than the refractive index of light guide body 12.

The light having entered light guide member 18 reaches the surface of light guide member 18 (“b” in FIG. 23), and is reflected by reflection film 20. The reflected light again enters light guide body 12 (“c” in FIG. 23). Then, the light having entered light guide body 12 is again reflected by back-facing surface 15 of light guide body 12 (“d” in FIG. 23), and again enters light guide member 18 (“e” in FIG. 23).

The light again having entered light guide member 18 reaches the boundary surface with finger joint 27 placed on projecting section 30 (“f” in FIG. 23), so that all the light is applied, in principle, to finger joint 27 without being totally reflected. This is because the refractive index of the biological tissue is equal to or higher than the refractive index of light guide member 18.

With the above-described configuration, even in the case of a patient with rheumatism accompanied by joint deformation, phototherapeutic treatment can be performed in the state where the bent part of the finger joint is brought into contact with light guide member 18 without requiring an unnatural action of the patient and without giving pain and suffering to the patient. Further, it is configured such that most of light having entered light guide body 12 is emitted from projecting section 30. Thereby, light can be efficiently supplied to the affected part which is placed on projecting section 30 and which is to be treated, and unnecessary emission of light to the outside from light guide body 12 can be prevented. As a result, efficient therapeutic treatment can be provided.

### Embodiment 4

Embodiment 4 has basically the same configuration as the configuration of phototherapy apparatus 1 in Embodiment 3, but has a configuration which can be more easily used by a user.

When phototherapeutic treatment is performed by using phototherapy apparatus 1 according to the present invention, a patient has to place his/her affected part on light guide body 12 and auxiliary light guide body 13 periodically for a certain period of time. Therefore, it is preferred that phototherapeutic treatment can be performed in a state comfortable for a patient with rheumatoid arthritis.

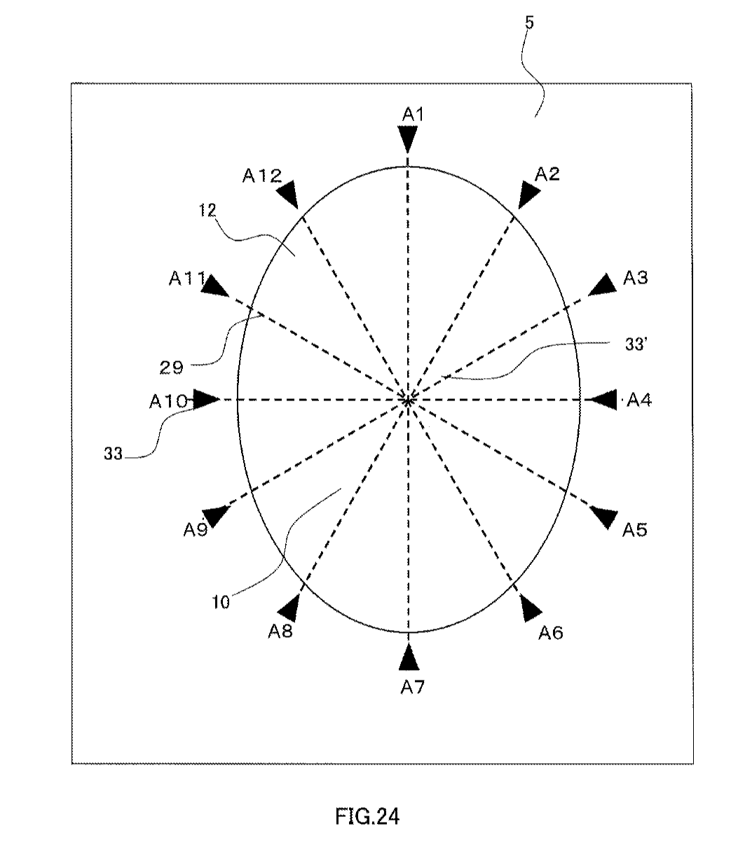

FIG. 24 is a top view of light guide body 12 disposed on base 5. As shown in FIG. 24, positioning indications 33 and positioning auxiliary lines 33′ for disposing auxiliary light guide body 13 are provided on base 5 around light guide body 12 of phototherapy apparatus 1 in Embodiment 4. Since light guide body 12 is transparent, positioning auxiliary lines 33′ can be visually checked from the upper surface of light guide body 12.

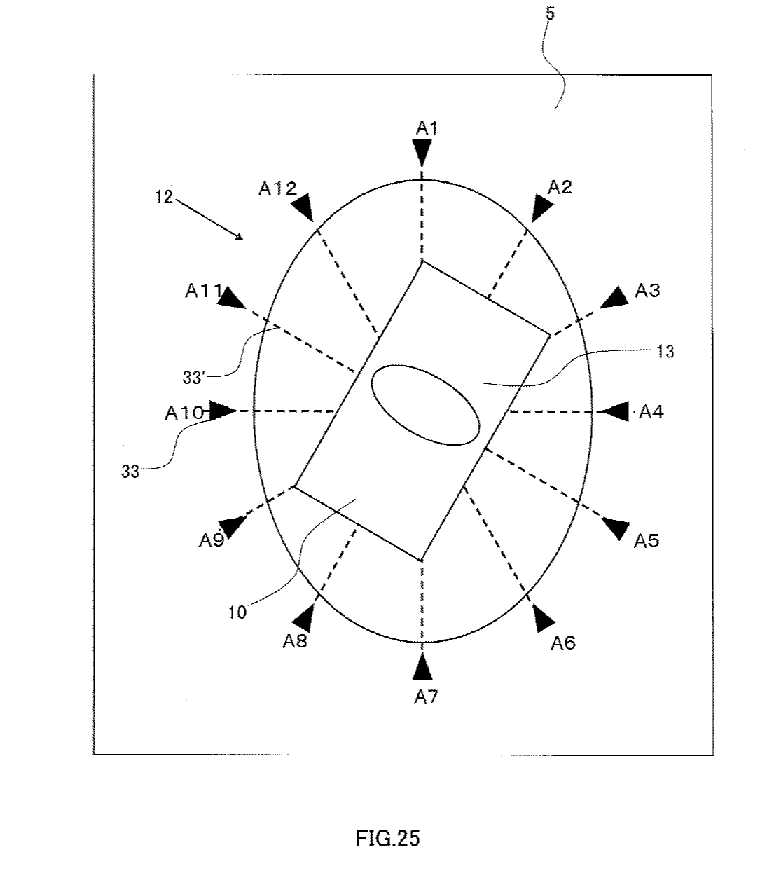

As shown in FIG. 25, auxiliary light guide body 13 is disposed on placement surface 14 of light guide body 12 so that positioning indications 33 and positioning auxiliary lines 33′ respectively coincide with the corner parts of auxiliary light guide body 13. For example, in the case where therapeutic treatment can be applied to a patient in a state most comfortable for the patient at the time when auxiliary light guide body 13 is disposed at the positions of A1, A3, A7, and A9, the patient needs only to grasp positioning indications 33. Then, each time the therapeutic treatment is applied to the patient, the therapeutic treatment may be performed by disposing auxiliary light guide body 13 so that the corner parts of auxiliary light guide body 13 coincide with the positions of A1, A3, A7 and A9 as shown in FIG. 25.

In this way, when positioning indications 33 and positioning auxiliary lines 33′ for disposing auxiliary light guide body 13 are provided on base 5 around light guide body 12 so as to be used as marks for determining the placement position of auxiliary light guide body 13, a patient can receive therapeutic treatment in a state comfortable for the patient. The patient can easily receive therapeutic treatment without the need of finely adjusting the placement of auxiliary light guide body 13 each time receiving the therapeutic treatment.

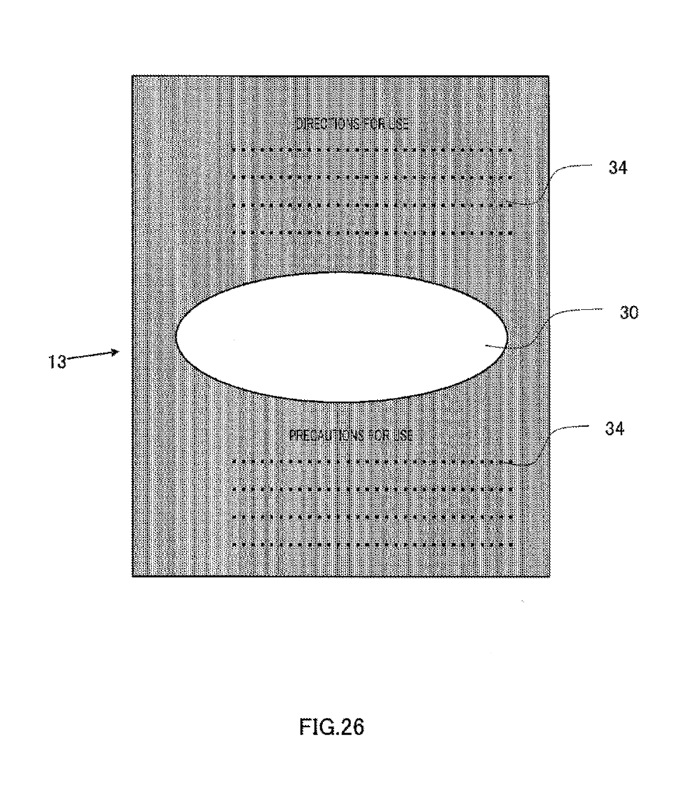

Note that, for example, when characters and figures indicating the method for disposing auxiliary light guide body 13 on light guide body 12, the method for using the phototherapy apparatus, precautions, and the like are printed (printed section 34) on reflection film 20 of auxiliary light guide body 13 as shown in FIG. 26, auxiliary light guide body 13 can be configured to be more easily used by a user.

### INDUSTRIAL APPLICABILITY

In the phototherapy apparatus according to the present invention, only by placing an affected part so that a treatment part is brought into contact with the light guide member disposed on the surface of the light guide body, the treatment part can be brought into contact with the light guide member. This is because the light guide member constituting the auxiliary light guide body has softness and hence is deformed according to the shape of the treatment part. Further, even when an affected part is a bent part, such as a finger joint, therapeutic treatment can be performed without requiring an unnatural action of a patient.

Further, light having entered the inside of the light guide body from a light source can be supplied to a treatment part via the light guide member of the auxiliary light guide body. As a result, sufficient therapeutic light can also be supplied to a bent part, such as a finger joint, and hence suitable therapeutic treatment can be performed.

Further, light can be intensively supplied to a treatment part by using the light guide member according to the present invention. For example, when a hand is assumed to be a target of treatment of rheumatism, the treatment part is a finger joint. In this case, use of the light guide member prevents light from being supplied to parts other than the finger joint. As a result, the energy efficiency of the phototherapy apparatus can also be improved. Further, even when therapeutic treatment needs to be intensively applied to the finger joint portion, therapeutic light can be highly efficiently supplied to the finger joint portion, and hence the improvement of the therapeutic efficacy can also be expected.

Further, in the phototherapy apparatus according to the present invention, since an inexpensive and general-purpose material, such as silicone resin, can be used as the material of the light guide member, the light guide member is suitable to be used as a disposable member, and hence therapeutic treatment in consideration of sanitation can be performed.

For this reason, the phototherapy apparatus according to the present invention is expected to be widely used as a phototherapy apparatus, for example, for treating a hand and finger joints of a patient with rheumatoid arthritis.

### REFERENCE SIGNS LIST

1 Phototherapy apparatus
2 Desk
3 Patient
4 Chair
5 Base
6 Lid
7 Hinge
8 insertion port
9 Engagement hole
10 Hook
11 Hook button
12 Light guide body
13 Auxiliary light guide body
14 Placement surface
15 Back-facing surface
16 Incident port
17 Light source
18 Light guide member
19 Holding member
20 Reflection film
21 Metacarpophalangeal joint
22 Proximal interphalangeal joint
23 Treatment part contact surface
24 Holding surface
25 Incident angle a
26 Incident angle b
27 Finger joint
28 Gap
29 Incident angle c
30 Projecting section
31, 32 Boundary
33 Positioning indication
33′ Positioning auxiliary line
34 Printed section

1. A phototherapy apparatus comprising:
a light guide body that includes a first surface on which an affected part is placed, and a second surface on a back surface side of the first surface;
a light source that outputs therapeutic light to enable the therapeutic light to enter an inside of the light guide body; and
a light guide member that has softness and surface tackiness and is disposed on a part of the first surface.

2. The phototherapy apparatus according to claim 1, wherein the material of the light guide member is an elastomer.

3. The phototherapy apparatus according to claim 1, wherein the light guide member is disposed at a position in contact with a treatment part to be irradiated with the therapeutic light, the treatment part being part of the affected part placed on the first surface.

4. The phototherapy apparatus according to claim 1, wherein the light guide member has a projecting shape with respect to the first surface.

5. The phototherapy apparatus according to claim 1, wherein the light guide member is configured to be detachable from the first surface.

6. The phototherapy apparatus according to claim 1, further comprising a holding member that has a hollow part therein, wherein
the light guide member is inserted and held in the hollow part.

7. The phototherapy apparatus according to claim 6, wherein the light guide member is held while being raised from the peripheral edge of the hollow part of the holding member.

8. The phototherapy apparatus according to claim 6, wherein the holding member includes a reflection film that is provided on the inner wall of the hollow part and that reflects the therapeutic light.

9. The phototherapy apparatus according to claim 1, wherein the light guide member is substantially sheet-shaped,
a back surface of the light guide member is a surface held on the first surface, and an obverse surface of the light guide member is a surface on which the affected part is placed, and
a projecting section projecting with respect to the first surface is provided on a part of the obverse surface that is brought into contact with a treatment part.

10. The phototherapy apparatus according to claim 9, wherein a reflection film that reflects light from the obverse surface toward the back surface is provided on a part of the obverse surface of the light guide member except the projecting section.

11. The phototherapy apparatus according to claim 9, wherein the projecting section is formed in a shape having a width in one direction and a width in another direction perpendicular to the one direction, and
the width in the one direction is larger than the width in the another direction.

12. The phototherapy apparatus according to claim 1, wherein the light guide body has a light guide path end surface provided with an incident port for receiving the therapeutic light from the light source, and
the light source is disposed at the incident port.

13. The phototherapy apparatus according to claim 1, wherein the light guide body has a shape of a hollow semi-spherical body, and the first surface is the outer surface of the hollow semi-spherical body.

14. The phototherapy apparatus according to claim 1, wherein the light guide body is disposed on a base, and
a positioning indication that enables the light guide member to be disposed at a desired position on the first surface is provided on the surface of the base.
